(19) United States
(12) Patent Application Publication
(10) Pub. No.: US 2015/0224279 A1
(43) Pub. Date: Aug. 13, 2015

(54) Medical Aerosol Device

(71) Applicant: Kenneth G. Miller, San Antonio, TX (US)

(72) Inventor: Kenneth G. Miller, San Antonio, TX (US)

(21) Appl. No.: 14/177,388

(22) Filed: Feb. 11, 2014

(51) Int. Cl. A61M 16/14; A61M 16/10; A61M 16/00

(52) U.S. Cl. CPC A61M 16/147; A61M 16/0096; A61M 16/0012; A61M 16/1005; A61M 16/109; A61M 2202/0208; A61M 2202/04; A61M 2205/70; A61M 2205/3673; A61M 2205/3368

(57) ABSTRACT

A medical aerosol device comprising a jet nebulizer within an aerosol chamber, a heating unit, and a liquid source. The jet nebulizer comprises a jet nozzle and an orifice in fluid communication with the liquid source, and functions by passing a jet of oxygen across the face of the orifice to draw liquid from the fluid source and through the orifice, where it is finely dispersed into an aerosol by the jet stream. The orifice includes an impinged portion curved to direct additional airflow across the orifice face, which is also canted away from the jet stream, which together results in a stronger and more consistent suction force on the liquid, and an aerosol in which at least 80% of the individual droplets are five microns or less. An improved heating control system and composition of a heating platen is also contemplated, as well as a method of use.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

1. Technical Field

The present disclosure relates generally to medical nebulizers. More particularly, the present disclosure relates to an apparatus and associated method for creation and vaporization of an aerosol with superior flow rate, droplet size distribution and temperature control attributes.

2. Related Art

Nebulizers have been used in the medical field for many decades to provide to therapeutic aerosols for inhalation by a patient. Typically, a nebulizer will rely on oxygen, compressed air, or ultrasonic waves to finely disperse a liquid into an aerosol, also known as a mist, made up of very small droplets. The resulting aerosol is then communicated to the patient via inhalation through the lungs. The liquid may be, for example, water or another liquid, or a liquid containing a medication in a solution or suspension.

Aerosols are very useful for treatment of respiratory diseases. For delivery of therapeutic aerosols to the lower respiratory tract, it is widely recognized that size plays an important role in determining where the aerosol particle will deposit once inhaled. The lungs, together with the airways of the respiratory tract, have evolved to form a particle size-selective system in which progressively finer particles are removed from inhaled air as they pass through the mouth, larynx, and bronchioles. It is generally accepted that the smaller the aerosolized particle, the further into the respiratory tract the particle will travel. Thus, it is important that for a nebulizer to be effective in producing aerosols that will reach a targeted area of the respiratory tract, it must produce droplets that are consistent in their size.

One type of nebulizer is a jet nebulizer, also called an atomizer. Jet nebulizers typically operate by passing a stream of compressed air or oxygen through a liquid to convert the liquid into an aerosol. Jet nebulizers are often useful for patients who require frequent or long-term access to therapeutic aerosols.

Some jet nebulizers rely on the Bernoulli effect to induce liquid to flow from a reservoir containing the liquid into the chamber in which it is converted into an aerosol. This may be achieved, for example, by passing the stream of compressed air or oxygen across the face of a tube connected to the fluid reservoir. The resulting pressure differential created by the higher rate of airflow outside the tube will result in liquid being drawn out from the reservoir, through the tube, and into the path of the jet flow, where it is converted into an aerosol.

However, jet nebulizers of this design suffer from some significant deficiencies. For example, the suction force in a jet nebulizer of this design may not be sufficiently strong enough or consistent enough to draw fluid from the reservoir at a sufficient or consistent volume, especially when the level of fluid in the reservoir may drop, requiring higher levels of suction force.

Likewise, many known jet nebulizers may not provide a sufficient volume of compressed air or oxygen to adequately nebulize the liquid as well as provide a desired level of air or oxygen to the patient, such as when the nebulizer is also used to augment or support the breathing of the patient.

Further, many jet nebulizers may produce aerosols in which the size distribution of the aerosol particles is not sufficiently controlled or consistent, resulting in the production of a large proportion of droplets having a size which do not meet the medical needs of the patient.

It is therefore desirable to provide a jet nebulizer which may provide a stronger and more consistent suction force, a greater volume of aerosol to the patient, and a more controlled and consistent size of aerosol droplet.

Jet nebulizers may also be paired with heating systems to heat the resulting aerosol prior to delivery to the patient. Such systems may include, for example, a heating platen which heats the aerosol prior to exiting the nebulization chamber.

However, such heating platens also suffer from significant deficiencies. For example, they may not maintain a consistent temperature when exposed to the aerosol environment of the nebulization chamber, and instead may be prone to temperature fluctuations. This may lead to aerosols of uneven temperatures being delivered to patients. Such uneven temperatures may result in adverse medical outcomes, for example, when a therapy may require that the liquid be delivered at, above, or below a certain temperature for optimal efficacy. Further, temperatures too low may result in insufficient heating of the aerosol, and temperatures too high may result in too much rainout of aerosolized liquid, leading to insufficient therapeutic effect, or even discomfort, pain, or injury to the patient if the aerosol is heated to excessive temperatures.

It is therefore also desirable to provide an aerosol heating platen which may heat an aerosol in a manner which is more even and resistant to temperature fluctuations.

These and other advantages are implemented in the present disclosure, as described in further detail below.

### BRIEF SUMMARY

To solve these and other problems, a medical aerosol device is contemplated in which the Bernoulli effect of the jet nebulizer is enhanced to result in the application of a stronger and more consistent suction on the liquid to be aerosolized and the resulting aerosol droplet size is more strictly controlled. Such enhancements are attributable to design features of the jet nozzle and liquid orifice. In particular, the face of the orifice may be canted away from the jet of airflow passing across it, resulting in the creation of a low pressure region intermediate the face of the orifice and the flow of air and thus a greater suction force on the liquid within the reservoir. In addition, the portion of the orifice facing the jet nozzle may be impinged upon and curved to cause the impinging air to flow across the face of the orifice, resulting in increased airflow across the face of the orifice and a stronger seal along the impinged portion, resulting in a more strongly defined pressure differential at the low pressure region. Further, the diameters of the jet nozzle and the orifice are regulated to achieve a desired volume of flow. Together, these features combine to result in an aerosol having a particle size distribution which is highly effective for traveling deep within the respiratory tract. Moreover, the heating platen of the medical aerosol device incorporates superior temperature control features, resulting in a more controllable and consistent temperature of the resulting aerosol.

The jet nebulizer may comprise a jet nozzle for delivering a gas, and an orifice for delivering a liquid to be converted into the aerosol. The orifice may be positioned such that liquid flowing through the orifice exits the orifice in a direction substantially transverse to the flow path of the gas exiting the jet nozzle, and such that the gas exiting the jet nozzle at least partially impinges upon a portion of the orifice facing the jet nozzle and at least partially passes across the face of the orifice. The face of the orifice may be canted away from the flow path of the gas exiting the jet nozzle. A low pressure region may thus be defined intermediate the face of the orifice and the path taken by the flow of gas across the face of the orifice, resulting in an improved suction of the liquid from the liquid source.

Preferably, the face of the orifice is canted away from the flow path of gas exiting the jet nozzle at an angle between 2½° and 5° relative to the axis of flow path of the gas exiting the jet nozzle. More preferably, the face of the orifice may be canted away from the flow path at a 4° angle relative to the axis of the path of the gas exiting the jet nozzle. Additionally, the impinged portion of the orifice may be at least partially curved so as to cause the impinging gas to remain in contact with the impinged portion and to cause at least a portion of the impinging flow of gas to cross the face of the orifice.

The jet nozzle may have at its throat an interior diameter of at least 0.042 inches, measured transverse to the flow path of the gas exiting the jet nozzle, and the orifice may have an interior diameter of at least 0.033 inches, measured transverse to the flow path of the liquid exiting the orifice. The gas flow rate through the jet nozzle may be at least 40 liters per minute. The gas delivered by the jet nozzle may be oxygen, and the liquid delivered by the orifice may be water. The liquid delivered by the orifice may also be a therapeutic compound. At least 80% of the individual droplets of the resulting aerosol may have a diameter of 5 microns or less.

The jet nebulizer may also have one or more entrainment apertures for entraining ambient air. The one or more entrainment apertures may have an adjustable area. The adjustability may be achieved via the use of an entrainment iris. The entrainment iris may be adjusted according to one or more calibration markings defining positions to which the entrainment iris may be adjusted so as to entrain one or more predetermined volumes of ambient air. The calibration markings may be embossed up on, molded in, or indicated by voids through the material of the entrainment iris. Preferably, the calibration marking are molded through the material of the entrainment iris to enhance visibility in dim light.

The heating platen of the medical aerosol device may be a thermally conductive plate having as its opposed surfaces a heating surface and a temperature control surface. A heating element may be disposed proximate the temperature control surface for heating the plate. Two thermistors may be disposed against the temperature control surface of the plate in a diametrically opposing configuration for determining a temperature of the vaporization surface from an amalgamation of the outputs of the thermistors. A thermal switch may be in communication with the thermistors and the heating element. The thermal switch may control the operation of the heating element in response to the outputs of the thermistors, to maintain the temperature of the plate within a desired temperature range. Preferably, the desired temperature range is between 100° F. and 106° F. The thermally conductive plate may include or be composed of elemental nickel.

A method for providing a heated aerosol is also contemplated, comprising generating an aerosol with the jet nebulizer according to an embodiment as described above, and positioning the platen, proximate the flow path of the resulting aerosol from the jet nebulizer, resulting in a heated aerosol.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a front view of one embodiment of a medical aerosol device in accordance with one embodiment of the present invention;

FIG. 2 is an exploded front view of an embodiment of the medical aerosol device;

FIG. 3 is a detailed cutaway front view of and embodiment of the medical aerosol device, showing the flow path of a generated aerosol;

FIG. 4 is a cross-sectional view of a jet nebulizer system according to one embodiment of the present invention;

FIG. 5 is a detailed cross-sectional view taken within circle 5 of FIG. 4, showing a detailed view of the jet nebulizer system according to one embodiment of the present invention;

FIG. 6 is a perspective view of an aerosol chamber of one embodiment of a medical aerosol device, including an entrainment control system;

FIG. 7 is a top view taken upon line 7 of FIG. 6, showing an embodiment with a fully open entrainment iris completely exposing the entrainment ports;

FIG. 8 is a top view taken upon line 8 of FIG. 6, showing an embodiment of the present invention with a partially open entrainment iris, partially exposing the entrainment ports; and

FIG. 9 is a top view taken upon line 9 of FIG. 6, showing an embodiment of the present invention with a nearly closed entrainment iris, only slightly exposing one entrainment port.

Common reference numerals are used throughout the drawings and the detailed description to indicate the same elements.

### DETAILED DESCRIPTION

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

According to various aspects of the present invention, an improved medical aerosol device is contemplated, which utilizes enhanced configurations of jet nebulizers, heating units, and air entrainment systems. It is contemplated that the jet nebulizer may include a jet nozzle and an orifice in fluid communication with a liquid source, which may be configured such that the gas exiting the jet nozzle partially flows across the face of the orifice, and partially impinges upon a portion of the orifice which is curved so as to direct the impinging airflow across the face of the orifice. The face of the orifice may be canted away from the flow path of the gas exiting the jet nozzle to define a low pressure region intermediate the face of the orifice and the flow of gas across the face of the orifice. It is additionally contemplated that the size of the jet nozzle throat and the orifice inner diameter may be configured to enhance aerosol creation. This configuration of jet nebulizer may result in improved suction of liquid from the liquid source. The heating unit may have a platen comprising elemental nickel, and may include a pair of thermistors diametrically opposed on the heating surface of the platen. This configuration of heating platen may result in a more controlled and stable heating of an aerosol. An entrainment system is further contemplated which may include one or more entrainment ports and an entrainment iris. The entrainment iris may include calibration marks which are embossed upon, molded in, or indicated by voids through the entrainment iris. This configuration of entrainment system may result in improved visibility, tactile feedback, endurance, and sterility.

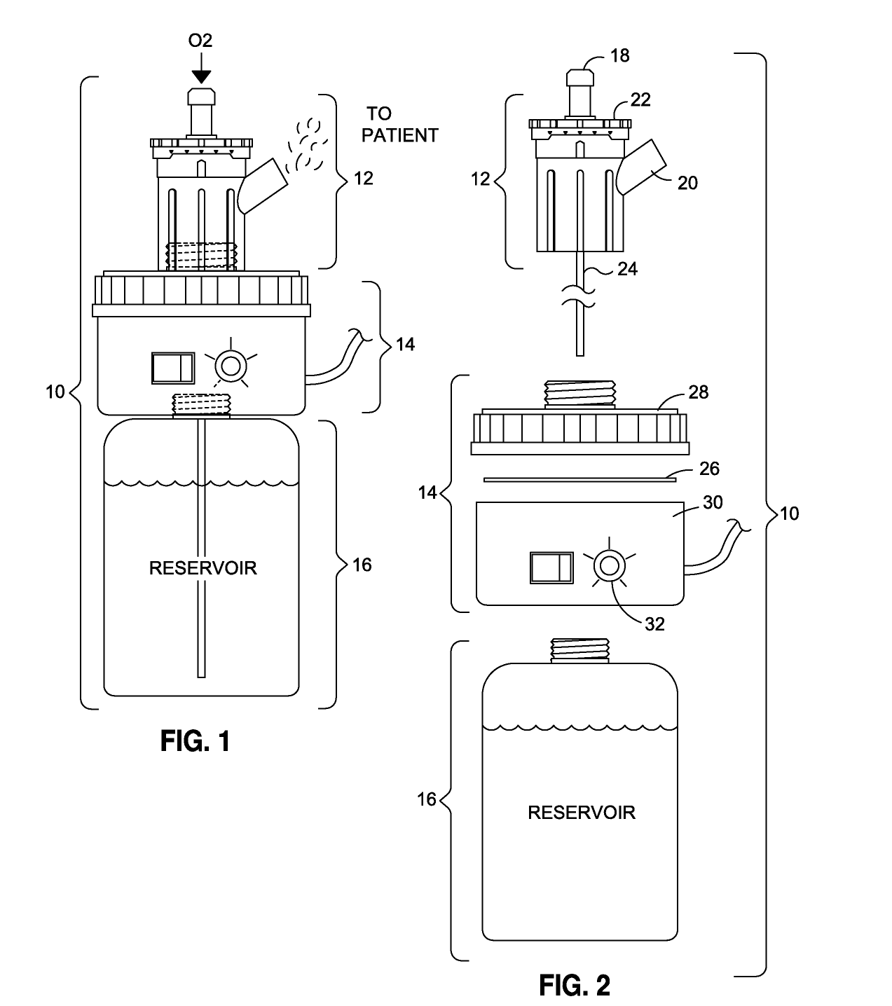

Referring now to the drawings, and more particularly to FIG. 1, a medical aerosol device 10 according to an exemplary embodiment of the present invention is shown. In such an exemplary embodiment, the medical aerosol device 10 may include an aerosol chamber 12, a heating unit 14, and a liquid source 16.

Referring now to FIG. 2, an exploded view of the exemplary embodiment of the medical aerosol device 10 is shown. It may be seen that the aerosol chamber 12 may interface with the heating unit 14, and the heating unit 14 may interface with the liquid source 16. It is preferable that the interfaces between these components be sealable to be airtight and watertight. Such interfacing may be achieved by, for example but without limitation, threaded engagement, frictional engagement, or coupling engagement. In the exemplary embodiment, the aerosol chamber 12 and the liquid source 16 are threadably engageable with the heating unit 14. However, it may be seen that other known forms of engagement, including those mentioned above, may be utilized, so long as the form of engagement generally results in an airtight and watertight seal when engaged, so as to mitigate leakage of gas, liquid, or aerosol, as well as mitigating potential introduction of external foreign substances which may cause contamination of the produced aerosol or damage to the integrity or performance of the interfaces or medical aerosol device 10 itself.

The aerosol chamber 12 and its subsidiary components may be formed of one or more materials usable in the making of medical devices, including, for example but without limitation, plastics, metals, or ceramics. In the exemplary embodiment, the aerosol chamber 12 is a cylindrical structure formed of high-density thermoplastic materials. The aerosol chamber 12 may have as its exterior components a gas intake port 18, an aerosol exit port 20, an entrainment system 22, and a suction tube 24.

The gas intake port 18 may be any port which allows intake of gas from a gas source. In the exemplary embodiment, such gas is oxygen at 50 PSIG, which is generally the standard operating pressure of a hospital flow meter. The gas intake port 18 may interface with the gas source by, for example but without limitation, threaded, frictional, or coupling engagement, or any other form of engagement known for coupling a gas source to a gas intake port which does not result in appreciable leakage or pressure loss when correctly coupled. In the exemplary embodiment, the interface between the gas intake port 18 and the gas source is achieved via threaded engagement of the gas intake port 18 to a conduit connected to the gas source.

The aerosol exit port 20 may be any port which allows output of aerosol produced by the medical aerosol device 10. In a medical setting, the aerosol exit port 20 may engage with an external system for transmission of the produced aerosol to a patient. Such engagement may be, for example but without limitation, threaded, frictional, or coupling engagement. In the exemplary embodiment, the aerosol exit port 20 is configured to allow for frictional engagement of, for example, a conduit which is sized and dimensioned to securely fit over or within the aerosol exit port 18, and which may be retained by elastic properties or another form of frictional locking device. However, it may be seen that other known forms of engagement, including those mentioned above, may be utilized.

The entrainment system 22 may be any entrainment system which allows for the introduction of gas into the aerosol chamber 12 via entrainment caused by the gas flowing into the aerosol chamber 12 from the gas intake port 18. The entrainment system 22 may be static so as to allow a static volume of air to be entrained by a gas having a given pressure and volumetric flow rate through the gas intake port 18, or the entrainment system 22 may be adjustable so as to allow a variable volume of air to be entrained by a gas having a given pressure and volumetric flow rate through the gas intake port 18. In the exemplary embodiment, the air entrainment system 22 is adjustable by the user or medical provider, so as to allow different volumes of air to be entrained into the aerosol chamber 12 by a gas having a given pressure and volumetric flow rate being introduced through the gas intake port 18.

The suction tube 24 may be sized and dimensioned to allow suction of liquid originating at the liquid source 16 to the jet nebulizer of the aerosol chamber 12. The suction tube 24 may be joined to and have its proximal end within the aerosol chamber 12 and, when the medical aerosol device 10 is fully assembled, may pass through the heating unit 14 and have its distal end open and disposed within the liquid source 16, where it may be positioned to intake liquid. It may be seen that the suction tube 24 may be formed of a material which is flexible, so as to allow for placement within a liquid sources 16 which may have a variety of shapes or sizes.

The heating unit 14 may have a heating platen 26, a heating unit cover 28, a heating unit body 30, and heating unit controls 32. The heating unit 14 may be engageable with the aerosol chamber 12 and the liquid source 16 via the methods of engagement previously described, and may allow for the passage therethrough of the suction tube 24.

The heating platen 26 may be a thermally conductive plate having as its opposing surfaces a heating surface and a temperature control surface. The heating surface may be the surface oriented to face the aerosol chamber 12, and the temperature control surface may be the opposing surface. The heating surface may be exposed to the path taken by the aerosol created by the jet nebulizer within the aerosol chamber 12 prior to the aerosol exiting the medical aerosol device 10 via the aerosol exit port 20, so as to provide the option of heating the resulting aerosol to a desired temperature. The heating platen 26 may be formed of a thermally conductive material, which may be, for example but without limitation, a metal or ceramic. Preferably, the material used should be both thermally conductive and resistant to degradation, such as by rusting, in the presence of oxygen and water, or other medically active substances or gases which may be desired to be delivered to a patient. In the exemplary embodiment, the heating platen 26 is a disc composed of elemental nickel, which has corrosion-resistance properties comparable to stainless steel, but a much higher rate of thermal conductivity. At 68° F., elemental nickel has a thermal conductivity of 52 Btu/(hr ° F. ft), while stainless steel, depending on its particular composition, has a thermal conductivity of between 7 and 26 Btu/(hr ° F. ft). The use of a platen material with high thermal conductivity may allow for improved control and uniformity of heating properties, which may result in the delivery of aerosols which are heated to a more consistent and precise temperature.

The heating unit cover 28 may be a member for interfacing the aerosol chamber 12 to the heating unit 14, and for providing an airtight and watertight seal between the two. Such interfaces may be achieved in fashions which are airtight and watertight, as substantially described above. The heating unit cover 28 may be formed of any material or materials usable in the making of medical devices, including, for example but without limitation, plastics, metals, or ceramics. In the exemplary embodiment, the heating unit cover 28 is formed of a rigid thermoplastic. Further, the heating unit cover may define in part the pathway of aerosol produced by the jet nebulizer and define an area for the aerosol to be placed in a thermal transfer relation with the heating platen 26 prior to exiting the medical aerosol device 10 via the aerosol exit port 20. In the exemplary embodiment, the heating unit cover 28 is a separate component from the aerosol chamber 12 and the heating unit body 30, and as such is threadedly engageable with both. However, it may be seen that in other embodiments, the heating unit cover 28 may be integrated with the aerosol chamber 12 or the heating unit body 30, and as such may not be readily disengageable from one, the other, or both. In embodiments in which the heating unit cover 28 is a separate component, such as the exemplary embodiment, it may be seen that such a configuration may allow the aerosol chamber 12 to be more readily reconfigured or replaced, and may allow the heating platen 26 to be more easily accessed. Reconfiguration or replacement of the aerosol chamber 12 may be useful, for example, in allowing for interfacing of the aerosol chamber 12 with different external components requiring different forms or sizes of interfaces, such as gas sources which may only interface with certain configurations of gas intake port 18 or aerosol outlets which may only interface with certain configurations of aerosol exit port 20, or to utilize a jet nebulizer or entrainment system 22 which results in an aerosol flow to a patient having different attributes, such as aerosol particle density or pressure and volumetric flow rate, or in order to replace a damaged or degraded aerosol chamber 12. Ready access to the heating platen 26 may also be useful in order to allow for its reconfiguration or replacement in the case of malfunction or degradation, or for cleaning or sterilization of the heating platen 26.

The heating unit body 30 may be a member for housing the heating platen 26 and the heating unit controls 32, and for interfacing with the liquid source 16. The heating unit body 30 may be formed of one or more materials usable in the making of medical devices, including, for example but without limitation, plastics, metals, or ceramics. In the exemplary embodiment, the heating unit body 30 is formed of high-density thermoplastic materials. The heating unit body 30 may serve to contain the heating platen 26, and preferably should be formed and configured to not be appreciably degraded or damaged by the heat produced thereby.

The heating unit controls 32 may be controls for allowing a user or medical provider to control the heating unit 14. In the exemplary embodiment, the heating unit controls 32 are physical controls comprising a power switch for controlling the on-off status of the heating unit 14, and a temperature control knob for adjusting the temperature to which it is desired to heat the aerosol produced by the medical aerosol device 10. In certain embodiments, however, it may be seen that the heating unit controls 32 may only consist of a power switch for embodiments in which the heating unit 14 is or may be calibrated to heat at a certain temperature. In other embodiments, the heating unit controls 32 may instead consist of software running on hardware within or separate from the medical aerosol device, for which inputs may be given as, for example, signals originating from user inputs on an input device such as a touchscreen located on the device, or signals from a user or medical provider input on a device separate from but in communication with the heating unit 14, with such signals being transmittable via a physical or wireless connection. For example, it may be appreciated that the heating unit controls 32 may be embodied within a control system that may regulate the other variable attributes of the medical aerosol device 10, such as gas flow rates. Such a control system may be located within the medical aerosol device 10, or external to the medical aerosol device 10, such as a computer program running on nearby terminal or handheld device, a hospital-wide network, or even a user's personal device such as a cellular telephone.

The liquid source 16 may be any container, sized and configured to interface with the other components of the medical aerosol device 10 such that liquid contained within the liquid source may be induced to flow from within the liquid source 16 into the suction tube 24 via the Bernoulli effect resulting from the jet nebulizer system within the aerosol chamber 12. Preferably, the liquid source 16, when ready for use with the medical aerosol device 10, is free from sources of potential contamination of the liquid contained within which will be converted to aerosol. In the exemplary embodiment, the liquid source 16 is a container attachable to the heating unit 14, in such a fashion that the suction tube 24 passes through the heating unit 14 and into the liquid source 16. It may be appreciated that attaching the liquid source 16 in such a fashion may help minimize the potential contamination of a resulting aerosol due to the suction tube 24 being contained within the sterile environment of the medical aerosol device. Thus any leaks in the suction tube 24 may result in lost efficiency, but will not result in the introduction of foreign materials, which may be possible if the suction tube 24 passes external to the medical aerosol device 10 to a liquid source 16 that does not attach to the medical aerosol device 10 as in the exemplary embodiment.

The liquid contained the liquid source 16 may be, for example but without limitation, water, another liquid, or a medicative substance dissolved in or in suspension in water or another liquid. It may thus be appreciated that when the liquid within the liquid source 16 has different properties, for example, density, viscosity, surface tension, or heat capacity, it may be necessary to alter the various other properties of the medical aerosol device in order to achieve a desired aerosol volume and particle density.

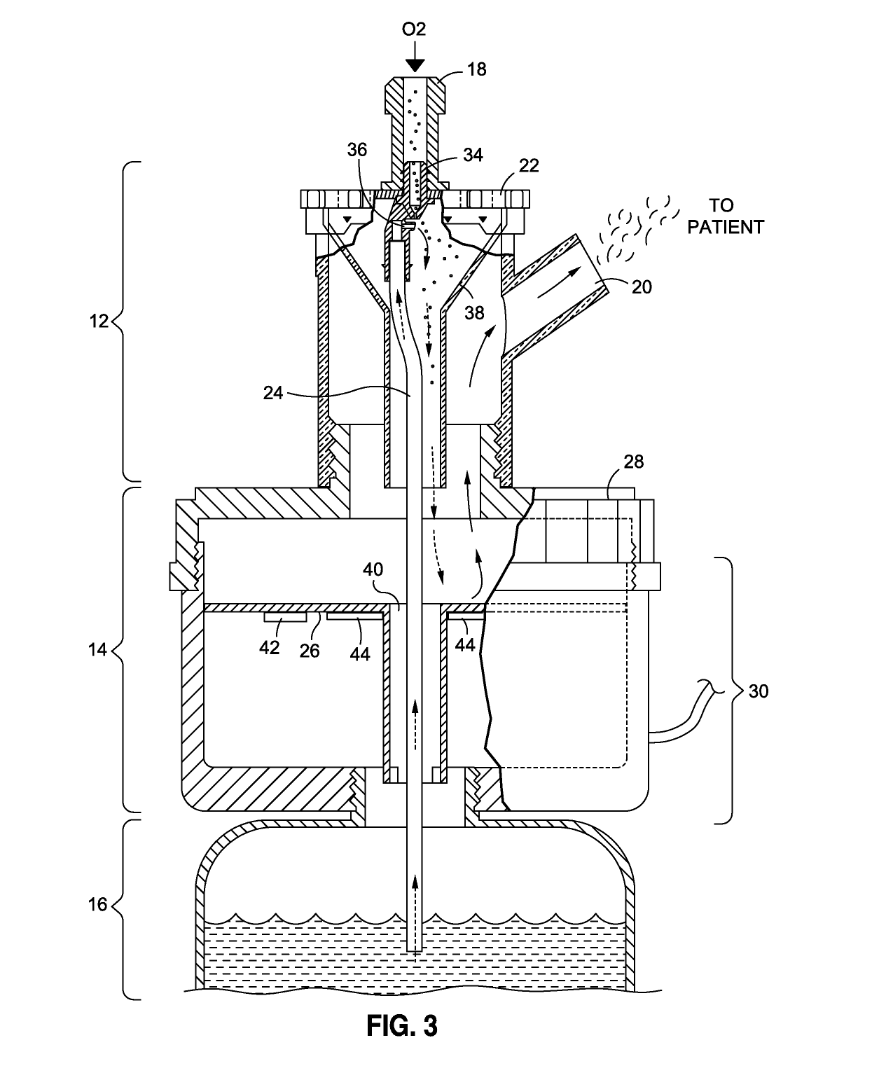

Referring now to FIG. 3, a detailed cutaway view of the medical aerosol device 10 is shown, illustrating the interior components and the flow path of the liquid and resulting aerosol produced by the jet nebulizer system. It may be seen that, according to the exemplary embodiment of the medical aerosol device 10, the aerosol chamber may further comprise a jet nozzle 34, an orifice 36, and an aerosol guide 38, and the heating unit may further comprise a rainout duct 40, a heating element 42, and thermistors 44.

The jet nozzle 34 of the jet nebulizer system within the aerosol chamber 12 may receive gas from the gas intake port 18 and direct it into the aerosol chamber 12. The jet nozzle 34 may control the flow attributes of the gas, and in the exemplary embodiment, convert it into a coherent stream, in order to induce liquid to flow up through the suction tube 24 via the Bernoulli effect, to disperse the liquid into an aerosol, and to cause ambient air to be entrained through the entrainment system 22. However, it may be seen that in other embodiments, the jet nozzle may perform only some of these functions, for example, in those embodiments without entrainment systems 22.

The orifice 36 of the jet nebulizer system is located at the proximal end of the suction tube 24 within the aerosol chamber 12 and may receive liquid flowing through the suction tube 24 from the liquid source 16 and emit the liquid into the flow path of the stream of gas from the jet nozzle 34, where it may be converted into an aerosol. The flow path of the stream of gas from the jet nozzle 34 additionally induces a Bernoulli effect at the orifice, which causes the liquid to be drawn up through the suction tube 24.

The aerosol guide 38 may be a structure within the aerosol chamber 12 which defines a flow path of the aerosol created at the jet nebulizer, and in particular causes the aerosol to flow proximal to the heating surface of the heating platen 26 prior to the aerosol being allowed to exit the medical aerosol device 10 via the aerosol exit port 20. In the exemplary embodiment, the aerosol guide 38 is, at its upper portion, conically tapered so as to contact the exterior walls of the cylindrical aerosol chamber 12, to position the openings of the entrainment system 22 within the aerosol guide 38. The aerosol guide 38 of the exemplary embodiment then tapers to converge at its lower portion in a narrower cylindrical structure at a point below the jet nebulizer system, to guide the produced aerosol downward in a flow path towards the heating surface of the heating platen 26, while also defining an area on the outside of the lower portion of the aerosol guide 38 for the aerosol to travel to the aerosol exit port 20 after the aerosol flow path takes the aerosol into proximity to the heating platen 26. In other embodiments, however, the aerosol guide 28 may be sized and configured differently, or not present at all. For example, in embodiments in which the entrainment system 22 may have openings at locations differing from that of the exemplary embodiment, the aerosol guide 38 may be sized and configured to place those openings on the interior of the aerosol guide where the ambient air may still be entrained by the gas from the jet nozzle 34.

It may be seen that the heating platen 26 may have a rainout duct 40, which is an aperture, opening, or passage through which liquid which has either not been converted to aerosol, or precipitated liquid that has rained out of the produced aerosol, may flow so that it does not accumulate on the surface of the heating platen 26. In the exemplary embodiment, the rainout duct is located in the center of the disc-shaped heating platen 26, and is a passage through the heating unit which is open on the other side to the liquid source. Additionally, the rainout duct 40 in the exemplary embodiment also serves the purpose of a providing passage through the heating unit 14 for the suction tube 24, allowing the suction tube 24 to be self-contained within the medical aerosol device 10. Thus, it may be seen that rained out liquid may be returned to the liquid source 16, reducing waste. Further, the liquid passing through the rainout duct 40 may also be heated due to proximity to the heating platen 26, resulting in warming of the liquid within the liquid source 16, and thus reducing the heat required to be transmitted to the aerosol by the heating platen 26 during extended use of the medical aerosol device 10. In other embodiments, however, the rainout duct 40 may not need to be located at the center of the heating platen 26, but may instead be located off-center, at an edge of the heating platen 26, or may even comprise an annular duct encircling the heating platen 26 and being further defined by the sides of the heating unit body 30. Further, while in the exemplary embodiment the heating platen 26 is flat on its heating surface, in other embodiments the heating surface of the heating platen 26 may be concave, convex, or otherwise irregularly shaped, so as to encourage rained out liquid to flow into a rainout duct 40 and not linger on the heating surface of the heating platen 26 where it may insulate aerosol from the heat from the heating platen 26 and reduce heating efficiency.

It may be seen that the temperature control surface of the heating platen 26, which opposes the heating surface, may contain one or more heating elements 42. It may be seen that the heating element may be any device which causes thermal energy to accumulate in the heating platen 26. The heating element 42 may be controlled by the heating unit controls 32 in combination with the thermistors 44. Suitable heating elements include, for example but without limitation, metallic, ceramic, or composite heating elements, or combinations thereof. In the exemplary embodiment, the heating element 42 comprises an etched ni-chrome element encapsulated in a formed mica/resin disc.

Additionally, the temperature control surface of the heating platen 26 may have one or more thermistors 44 for detecting the temperature of the heating platen 26. It may thus be seen that the use of the one or more thermistors 44 in combination with heating elements 42 and a thermal switch coordinated by the heating unit controls 32 may allow for the temperature of the heating surface of the heating platen 26 to be dynamically controlled in response to fluctuations due to certain factors, one of the most significant being fluid droplets striking the heating surface of the platen, requiring more heat. In the exemplary embodiment, it may be seen that by using two thermistors 44 diametrically opposed to each other on the temperature control surface of the heating platen in coordination with the heating unit controls, one or more thermal switches, and one or more heating elements 42, the temperature of the heating platen 26 may be more precisely controlled and refined, and better insulated from temperature fluctuations on different sides or locations of the heating surface.

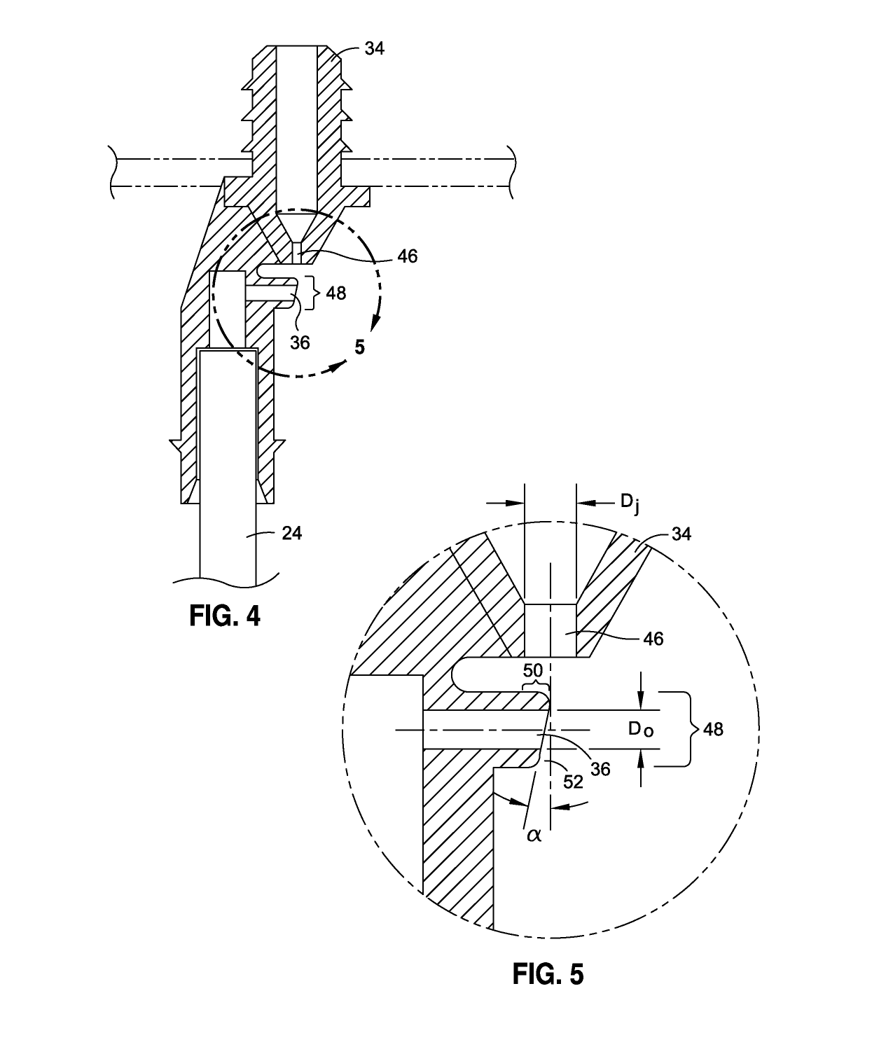

Referring now to FIG. 4, a detailed cross-sectional view of a jet nebulizer according to the exemplary embodiment is shown. It may be seen that the jet nozzle 34 has a jet nozzle throat 46, and the orifice has an orifice face 48.

Referring now to FIG. 5, a close-up cross-section view of the exemplary jet nebulizer of FIG. 4, taken within circle 5, is shown. In this close-up view, it may be seen that the jet nozzle throat 46 may have a jet nozzle throat diameter Dⱼ, the interior of the orifice 36 may have a orifice interior diameter Dₒ, the orifice may have an impinged portion 50 which is impinged upon by the stream of gas from the jet nozzle 34, and the orifice face 48 may be canted away from the flow of gas from the jet nozzle at an angle α, which results in a low pressure region 52 being defined intermediate the orifice face 48 and the flow of gas from the jet nozzle passing in front of the orifice face 48.

The jet nozzle throat 46 may be the distal tip of the jet nozzle 34 which defines the direction and cross-sectional area of the stream of gas exiting the jet nozzle 34 and travelling towards the orifice 36 which supplies the liquid to be converted to an aerosol. In the exemplary embodiment, the jet nozzle throat 46 directs gas downward towards the orifice 34, at an angle substantially transverse to the liquid flow path as the liquid exits the orifice 34.

The jet nozzle throat 46 may have a jet nozzle throat diameter Dⱼ. It is contemplated that in the jet nebulizer according to the exemplary embodiment, the jet nozzle throat diameter Dⱼ may be much wider than typical jet nebulizers, in order to allow for a greater volumetric flow rate of oxygen from a typical hospital flow meter in order to meet inspiratory demand. Most hospital flow meters typically operate at around 50 PSIG of pressure, which when used with a nebulizer having a typical jet nozzle throat diameter Dⱼ of around 0.020 inches, results in approximately 12 LPM of oxygen flow. However, this may be an excessive or insufficient amount of oxygen to meet inspiratory demand for patients requiring certain oxygen concentrations. Thus, it may be seen that the jet nozzle throat diameter Dj is preferably variable so as to regulate volumetric flow rates of gas passing therethrough, given a gas at a constant pressure. For example, for many patients with respiratory illness, a minimum of 40 LPM of oxygen may be required to meet that patient's inspiratory demand. Thus, it may be seen that in the exemplary embodiment, the jet nozzle throat diameter is at least 0.042 inches is provided to allow for the minimum 40 LPM to be delivered to the patient.

Further, it may be seen that the orifice face 48 may be canted away from the stream of gas at an angle α, so as to define a low pressure region 52 intermediate the orifice face 48 and the stream of gas crossing the orifice face. The defined low pressure region 52 results in an additional increase of the suction forces caused by the Bernoulli Effect. In the exemplary embodiment, the angle α of the cant of the orifice face 48 is at an angle of approximately 4° relative to the axis of the path of the gas exiting the jet nozzle 34. That construction of the exemplary embodiment, in combination with other above mentioned features of the jet nebulizer, and the use of oxygen at 50 PSIG and water as the liquid source, has been found to result in the production of a high-quality aerosol in which at least 80% of the individual droplets have a size of five or less microns. In other embodiments in which an improvement in suction force and aerosol particle size distribution may result, however, the cant angle α of the orifice face 48 may be been between 2½° and 5° relative to the axis of the path of the gas exiting the jet nozzle.

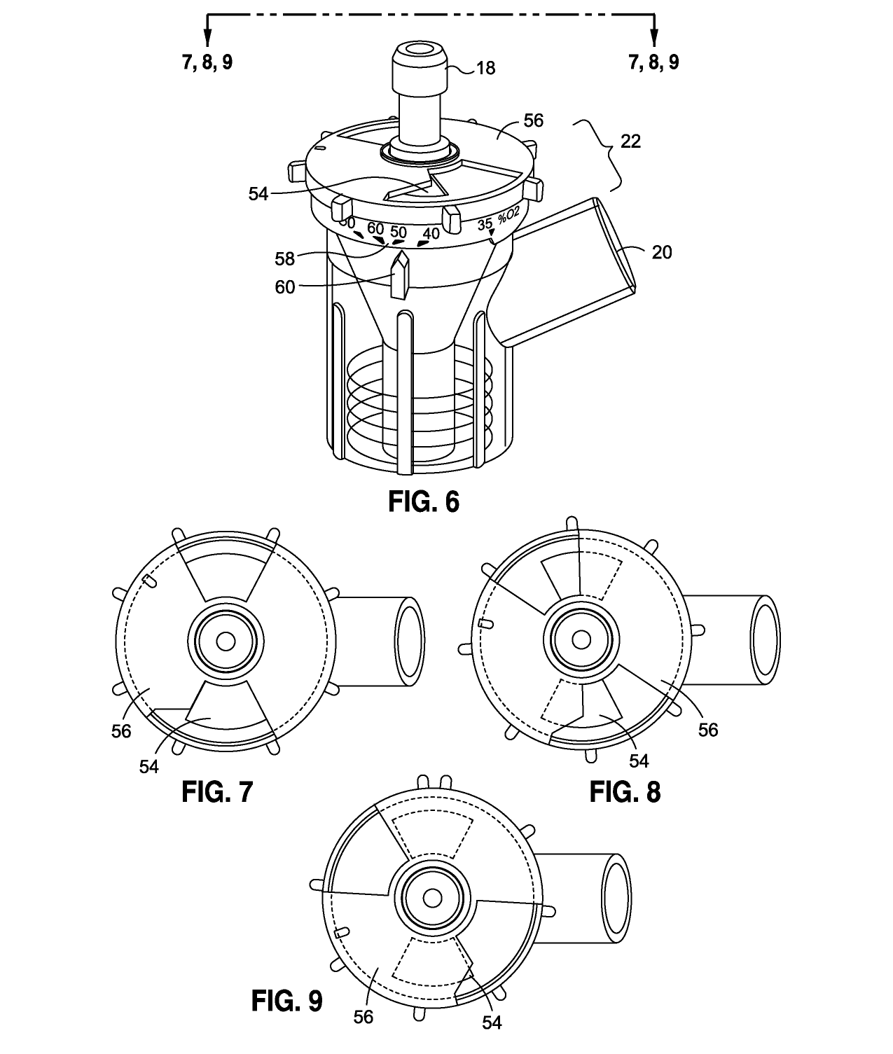

Referring now to FIG. 6, a perspective view of an aerosol chamber 12 having an entrainment system 22 is shown. It may be seen that an entrainment system 22 may have entrainment ports 54 and an entrainment iris 56 which may be adjusted by aligning calibration marks 58 with a calibration notch 60.

Entrainment port 54 may be any opening which is open to entry of ambient airflow via entrainment by the air stream from jet nozzle 34. Entrainment ports 54 may be sized and configured to entrain more or less air, as is desired, which may be achieved by sizing the entrainment ports differently on individual aerosol chambers, or by providing a mechanism for adjusting the exposed size of the entrainment ports 54. In the exemplary embodiment, an entrainment iris 56 is utilized to allow the user or the medical provider to adjust the size of the entrainment ports exposed to ambient air, in order to adjust the resulting volumetric flow rate of gas to the patient. The entrainment iris 56 may be situated atop the aerosol chamber 12 and may be rotatable to partially or even completely occlude the entrainment ports 54. In the exemplary embodiment, one of the cutouts of the entrainment iris 56 includes a wedge portion extending outward from the midpoint of one side of the cutout and towards the periphery of the entrainment iris 56, which allows the entrainment iris 56 to completely occlude the entrainment ports 54, except for a small portion of an entrainment port 54 which is not covered by the wedge portion. Such a configuration allows for adjustments of the entrainment iris 56 to configurations in which very small amounts of ambient air may be entrained through the entrainment ports 54, and in which minute adjustments may be made. This may be useful when, for example, high volumetric flow rates of oxygen are utilize and a small amount of entrainment of ambient air is desired, but exposure of more than miniscule portions of the entrainment ports 54 to ambient air may result in excessive ambient entrainment and too much dilution of oxygen, or even potentially dangerous overpressure conditions.

The entrainment iris 56 may have a series of calibration marks 58 for alignment with a calibration notch 60. Such calibration marks 58 may allow for simple regulation of the amount of air entrained and provided to the patient when used with predetermined gas input levels. For example, the exemplary embodiment shown in FIG. 6 may be adjusted by aligning its calibration markings 58 with the calibration notch 60 according the criteria of the below Table 1 to achieve certain volumes of air entrainment when oxygen gas at certain volumetric flow rates is connected to the gas intake port. However, in other embodiments having variations in entrainment systems 22, jet nozzle throat 46 size, or when used with gas sources of different pressures, the system of calibration may be substantially different from that of the exemplary embodiment.

TABLE 1

| Dial Setting | O₂ | Air | Total |
|---|---|---|---|
| (% O₂) | LPM | LPM | LPM |
| 36 | 14 | 60 | 74 |
| 40 | 15 | 47 | 62 |
| 50 | 15 | 26 | 41 |
| 60 | 15 | 15 | 30 |
| 80 | 44 | 16 | 60 |
| 95 | 44 | 3 | 47 |

Referring now to FIG. 7, it may be seen that in the exemplary embodiment, when the entrainment iris 56 is turned to the 35% O₂ position, the entrainment ports 54 are fully exposed, allowing the maximum amount of entrainment of ambient air into the aerosol chamber 12.

Referring now to FIG. 8, it may be seen that in the exemplary embodiment, when the entrainment iris 56 is turned to the 40% position, the entrainment ports 54 are partially exposed, allowing a moderate amount of entrainment of ambient air into aerosol chamber 12.

Referring now to FIG. 9, it may be seen that in the exemplary embodiment, when the entrainment iris 56 is turned to the 80% position, only a small portion of one entrainment port 54 is exposed by the wedge portion in the entrainment iris 56, allowing only a very small amount of entrainment of ambient air into the aerosol chamber 12.

The above description is given by way of example, and not limitation. Given the above disclosure, one skilled in the art could devise variations that are within the scope and spirit of the invention disclosed herein, including various ways of configuring the aerosol chamber 12, the heating unit 14, and the liquid source 16. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A jet nebulizer for creating an aerosol, the jet nebulizer comprising:
a jet nozzle in fluid communication with a gas source for delivering a gas; and
an orifice defining an orifice face, the orifice being in fluid connection with a liquid source for delivering a liquid to be converted into the aerosol, the orifice being positioned such that liquid flowing through the orifice exits in a direction substantially transverse to a gas flow path exiting the jet nozzle, and such that the gas flow path at least partially impinges upon a portion of the orifice facing the jet nozzle and at least partially passes across the orifice face;
wherein the face of the orifice is canted away from the gas flow path of gas exiting the jet nozzle, defining a low pressure region intermediate the face of the orifice and the gas flow path taken across the face of the orifice, resulting in an increased suction of the liquid from the liquid source.

2. The jet nebulizer of claim 1, wherein the face of the orifice is canted away from the flow path of gas exiting the jet nozzle at an angle between 2½° and 5° relative to the axis of flow path of the gas exiting the jet nozzle.

3. The jet nebulizer of claim 1, wherein the face of the orifice is canted away from the flow path of gas exiting the jet nozzle at a 4° angle relative to the axis of the path of the gas exiting the jet nozzle

4. The jet nebulizer of claim 1, wherein the impinged portion of the orifice is at least partially curved so as to cause the impinging gas to remain in contact with the impinged portion and to cause at least a portion of the impinging flow of gas to cross the face of the orifice.

5. The jet nebulizer of claim 1, wherein the jet nozzle has at its throat an interior diameter of at least 0.042 inches measured transverse to the flow path of the gas exiting the jet nozzle, and the orifice has an interior diameter of at least 0.033 inches measured transverse to the flow path of the liquid exiting the orifice.

6. The jet nebulizer of claim 1, wherein the gas flow rate through the jet nozzle is at least 40 liters per minute.

7. The jet nebulizer of claim 1, wherein the gas delivered by the jet nozzle comprises oxygen.

8. The jet nebulizer of claim 1, wherein the liquid delivered by the orifice is water.

9. The jet nebulizer of claim 1, wherein the liquid delivered by the orifice comprises a therapeutic compound.

10. The jet nebulizer of claim 1, wherein at least 80% of the individual droplets of the created aerosol have a diameter of 5 or less microns.

11. The jet nebulizer of claim 1, further comprising one or more entrainment apertures for entraining ambient air.

12. The jet nebulizer of claim 11, wherein the one or more entrainment apertures has an adjustable area.

13. The jet nebulizer of claim 12, wherein the area of the one or more entrainment apertures are adjustable via an entrainment iris.

14. The jet nebulizer of claim 13, wherein the one or more entrainment apertures may be adjusted according to one or more calibration markings defining positions to which the entrainment iris may be adjusted so as to entrain one or more predetermined volumes of ambient air.

15. The jet nebulizer of claim 14, wherein the calibration markings are marked on the entrainment iris.

16. A platen having superior thermal stability attributes for heating an aerosol, the platen comprising:
a thermally conductive plate, the plate having as its opposed surfaces a heating surface and a temperature control surface;
a heating element disposed proximate the temperature control surface for heating the plate;
two thermistors disposed against the temperature control surface of the plate in an diametrically opposing configuration for determining a temperature of the vaporization surface from an amalgamation of the outputs of the thermistors; and
a thermal switch in communication with the thermistors and the heating element;
wherein the thermal switch controls the operation of the heating element in response to the outputs of the thermistors to maintain the temperature of the plate within a desired temperature range.

17. The platen of claim 17, wherein the desired temperature range is between 100° F. and 106° F.

18. The platen of claim 18, wherein the thermally conductive plate comprises elemental nickel.

19. The platen of claim 19, wherein the thermally conductive plate is composed of elemental nickel.

20. A method for providing a heated aerosol, the method comprising:
generating an aerosol with a jet nebulizer, the jet nebulizer comprising:
a jet nozzle in fluid communication with a gas source for delivering a gas; and
an orifice defining an orifice face, the orifice being in fluid communication with a liquid source for delivering a liquid to be converted into the aerosol, the orifice being positioned such that liquid flowing through the orifice exits in a direction substantially transverse to a gas flow path exiting the jet nozzle, and such that the gas flow path at least partially impinges upon a portion of the orifice facing the jet nozzle and at least partially passes across the orifice face;
wherein the face of the orifice is canted away from the flow path of gas exiting the jet nozzle, defining a low pressure region intermediate the face of the orifice and the gas path across the face of the orifice, resulting in an increased suction of the liquid from the liquid source; and
positioning a platen proximate a resulting aerosol flow path, the platen comprising:
a thermally conductive plate, the plate having as its opposed surfaces a heating surface and a temperature control surface;
a heating element disposed proximate the temperature control surface for heating the plate;
two thermistors disposed against the temperature control surface of the plate in an diametrically opposing configuration for determining a temperature of the vaporization surface from the outputs of the thermistors; and
a thermal switch in communication with the thermistors and the heating element;
wherein the thermal switch controls the operation of the heating element in response to the outputs of the thermistors to maintain the temperature of the plate within a desired temperature range.
